(19) United States
(12) Patent Application Publication
(10) Pub. No.: US 2008/0132910 A1
(43) Pub. Date: Jun. 5, 2008

(54) Control for a Remote Navigation System

(76) Inventor: Carlo Pappone, Milano (IT)

Correspondence Address: Edward H. Renner, Suite 400, c/o 7700 Bonhomme, St. Louis, MO, 63105, US

(21) Appl. No.: 11/874,892

(22) Filed: Oct. 18, 2007

Related U.S. Application Data
(60) Provisional application No. 60/864,789, filed on Nov. 7, 2006.

(51) Int. Cl. A61B 19/00

(52) U.S. Cl. 606/130

(57) ABSTRACT

A method of operating a remote navigation system comprising an orientation system for orienting the distal end of a medical device, and an advancing system for advancing and retracting the medical device. The method includes operating an input device to manipulate a cursor on the screen of a graphical user interface to operate the orientation system to orient the distal end of the medical device in a selected direction; and operating a track wheel on the input device to operate the advancing system to advance or retract the distal end of the medical device in the selected direction. A system for operating a remote navigation system comprising an orientation system for orienting the distal end of a medical device, and an advancing system for advancing and retracting the medical device. The system includes a display; a processor programmed to run a graphical user interface on the display for controlling the orientation system, and an input device for operating a cursor on the display to operate the graphical user interface to orient the distal end of the medical device in a selected direction; the input device having a track wheel for operating advancing system to advance and retract the distal end of the medical device in the selected direction.

### CROSS-REFERENCE TO RELATED APPLICATION

This application claims priority to prior U.S. Provisional Patent Application Ser. No. 60/864,789, filed Nov. 7, 2006, the entire disclosure of which is incorporated herein by reference.

### BACKGROUND

This invention relates to remote navigation systems for remotely orienting and advancing and retracting the distal end of a medical device such as an endoscope, a catheter, or a guide wire, in the body, and in particular to a control for such a remote navigation system.

Remote navigation systems have been developed that allow a user to remotely orient the distal end of a medical device in a selected direction in the body, and even advance and retract the device in the selected direction. Some of these systems use mechanical linkages such as pull wire or push wires, or combinations of pull wires and push wires; others of these systems use motors and gears or pulleys. Some of these systems employ magnetic navigation in which the distal end of the medical device is oriented by the application of a magnetic field in a selected direction by one or more source magnets, to orient a magnetically responsive element associated with the distal end of the medical device. Still other systems employ magnetostrictive, electrostrictive, or other shape changing elements to orient the distal end of a medical device.

The capabilities of these remote medical navigation systems have advanced to the point where a limiting factor in their use is the ability of the user to quickly, easily, and accurately provide inputs on the desired orientation and position of the medical device. While most of these remote navigation systems are computer controlled, controlling the many variables can be a daunting task, particularly for a new user.

### SUMMARY

The present invention relates to controls for remote navigation systems. These systems typically include orientation systems for controlling the orientation of the medical device and an advancer system for advancing and retracting the medical device. Embodiments of this invention provide a system and method for controlling remote navigation systems that allow the user to more easily operate a remote navigation system to control the position and orientation of a medical device.

In accordance with a preferred embodiment of this invention, a system is provided in which a user can operate a remote navigation system to control the orientation of the distal end of a medical device using a mouse or similar input device to move a cursor and click on a graphical user interface to selected the direction of orientation of the medical device, and the user can advance and retract the distal end of the medical device by operating a track wheel on the input device.

In accordance with another preferred embodiment of this invention, a method is provided in which a user can operate a remote navigation system to control the orientation of the distal end of a medical device using a mouse or similar input device to move a cursor and click on a graphical user interface to selected the direction of orientation of the medical device, and the user can advance and retract the distal end of the medical device by operating a track wheel on the input device.

The various embodiments of the systems and methods of the invention provide fast, simple and accurate control of a remote navigation system. These and other features and advantages will be in part apparent, and in part pointed out hereinafter.

### BRIEF DESCRIPTION OF THE DRAWINGS

FIG. 1 is a schematic diagram of a remote navigation system and computer control, with which embodiments of the present invention can be used;

FIG. 2 is a perspective view of a mouse with a track wheel of a type useful in the implementation of at least some embodiments of the present invention; and

FIG. 3 is a schematic diagram of a portion of a subjects; vasculature, illustrating a semi-automatic mode of navigation enabled by some embodiments of the systems and methods of the present invention.

Corresponding reference numerals indicate corresponding parts throughout the several views of the drawings.

### DETAILED DESCRIPTION

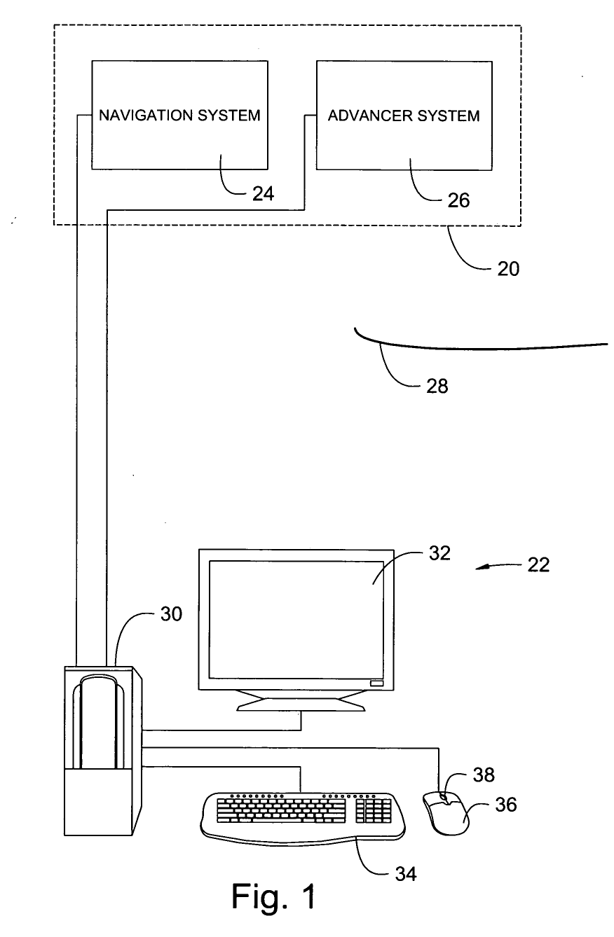

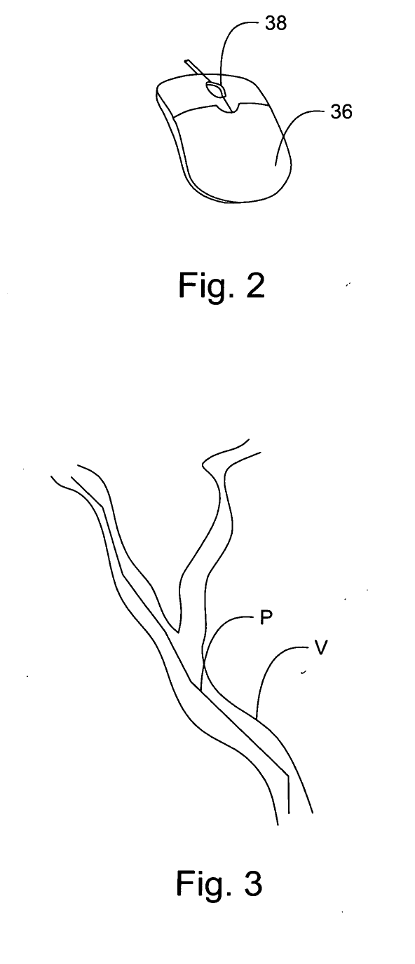

A remote navigation system 20 and its associated computer control system 22 are shown schematically in FIG. 1. The remote navigation system 20 comprises an orientation system 24 and an advancement system 26. The orientation system is preferably a magnetic navigation system of the type available from Stereotaxis, Inc., St. Louis, Mo. Examples of such systems are disclosed in U.S. Pat. No. 6,241,671, issued Jun. 5, 2001, for Open Field System for Magnetic Surgery; and U.S. Pat. No. 6,975,197, issued Dec. 13, 2005, for Rotating And Pivoting Magnet For Magnetic Navigation, the disclosures of which are incorporated by reference. However, the invention is not limited to magnetic navigation systems, and can be employed with any other type of system for remotely orienting the distal end of a medical device, for example mechanically operated systems, hydraulically or pneumatically operated systems, systems with shape changing elements such as electrostrictive or magnetostrictive elements, etc. The advancement system 26 advances and retracts the medical device. Examples of such systems are disclosed in U.S. Published Application No. 20060041245 for Systems And Methods For Medical Device Advancement And Rotation, and U.S. Published Application No. 20020177789 for System And Methods For Advancing A Catheter, the disclosures of which are incorporated herein by reference. While it is desirable that the system can advance and retract the medical device, it is possible that the system only operates in one direction, for example to advance the medical device.

The remote navigation system 20 is adapted for controlling the distal end of an elongate medical device 28. The medical device may be an endoscope, a catheter, a guide wire or other elongate flexible device that is adapted for navigation through the body, either through a body lumen or through the tissue.

The computer control system 22 comprises a central processor 30, which is operatively connected to both the orientation system 24 and the advancement system 26. The computer control system 22 comprises a display 32, and a keyboard 34 and a mouse 36, which preferably has a track wheel. Of course some other input device could be provided instead of mouse 36, provided that the input device has a track wheel or similar type of control.

The processor 30 runs graphical user interface software that is displayed on display 32. Examples of such interfaces are disclosed in Published Application No. 20060100505 for Surgical Navigation Using A Three-Dimensional User Interface; Published Application Nos. 20060041181, 20060041180, 20060041179, and 20060041178 for User Interface For Remote Control Of Medical Devices; Publication No. 20060036213 for Navigation Of Remotely Actuable Medical Device Using Control Variable And Length; Publication No. 20060036163 for Method Of, And Apparatus For, Controlling Medical Navigation Systems; Publication No. 20060036125 for User Interface For Remote Control Of Medical Devices; Publication No. 20060025719 Navigation Of Remotely Actuable Medical Device Using Control Variable And Length; Publication No. 20060025679 for User Interface For Remote Control Of Medical Devices; Publication No. 20060025676 for Navigation Of Remotely Actuable Medical Device Using Control Variable And Length; 18 20060025675 Navigation Of Remotely Actuable Medical Device Using Control Variable And Length; Publication No. 20060009735 for Navigation Of Remotely Actuable Medical Device Using Control Variable And Length; Publication No. 20050113812 for User Interface For Remote Control Of Medical Devices, the disclosures of all of which are incorporated herein by reference.

The user uses the mouse 36 to move a cursor on the display 32 and clicks the mouse to operate the interface to operate the orientation system 24, causing the orientation system 24 to orient the distal end of the medical device in a selected direction. The user can operate a track wheel 38 on the mouse 36 to operate the advancement system 26 to advance and retract the distal end in the selected direction. Thus, with a single input device a user can both orient or steer and advance and retract a medical device.

In one preferred mode, a localization system is provided for determining the position of the distal end of the medical device. As shown in FIG. 3, the vasculature V can be characterized, and a path P can be identified through the vasculature, as disclosed, for example in U.S. Published Application 20060074297 for Methods And Apparatus For Steering Medical Devices In Body Lumens. Then, as the device is advanced or retracted by operating the track wheel 38 on the mouse 36, the location can be determined, and the desired device orientation for that location is automatically determined. The orientation system 24 can be operated to automatically orient the device in the determined orientation for a given position, so that the device can be advanced along a preplanned path simply by operating the track wheel 38, without having to separately adjust the orientation of the distal end of the medical device.

What is claimed is:

1. A method of operating a remote navigation system comprising an orientation system for orienting the distal end of a medical device, and an advancing system for advancing and retracting the medical device, the method comprising:
operating an input device to manipulate a cursor on the screen of a graphical user interface to operate the orientation system to orient the distal end of the medical device in a selected direction; and
operating a track wheel on the input device to operate the advancing system to advance or retract the distal end of the medical device in the selected direction.

2. The method according to claim 1 wherein the input device is a mouse.

3. A method of navigating the distal end of a medical device through the body by operating a remote navigation system comprising an orientation system for orienting the distal end of a medical device, and an advancing system for advancing and retracting the medical device, the method comprising:
operating the advancing system in response to operation of the track wheel on an input device to move the distal end of the distal device through the body;
determining the position of the distal end of the device as it advances, and automatically operating the orientation system to cause the distal end to follow a preplanned path through the body.

4. The method according to claim 1 wherein the input device is a mouse.

5. A method of operating a remote navigation system comprising an orientation system for orienting the distal end of a medical device, and an advancing system for advancing and retracting the medical device, the method comprising: using a computer mouse to point and click a cursor on a graphical user interface to cause the orientation system to orient the distal end of the device in a selected direction, and operating a track wheel on the mouse to cause the advancing system to advance or retract the distal end of the device in a selected direction.

6. A system for operating a remote navigation system comprising an orientation system for orienting the distal end of a medical device, and an advancing system for advancing and retracting the medical device, the system comprising:
a display;
a processor programmed to run a graphical user interface on the display for controlling the orientation system, and
an input device for operating a cursor on the display to operate the graphical user interface to orient the distal end of the medical device in a selected direction; the input device having a track wheel for operating advancing system to advance and retract the distal end of the medical device in the selected direction.

7. The system according to claim 6 wherein the input device is a mouse.
